(19) United States
(12) Patent Application Publication
(10) Pub. No.: US 2022/0367052 A1
(43) Pub. Date: Nov. 17, 2022

(54) NEURAL NETWORKS WITH FEEDFORWARD SPATIAL TRANSFORMATION UNITS

(71) Applicant: Google LLC, Mountain View, CA (US)

(72) Inventors: Hanxiao Liu, Santa Clara, CA (US); David Richard So, San Francisco, CA (US); Quoc V. Le, Sunnyvale, CA (US); Zihang Dai, Pittsburgh, PA (US)

(21) Appl. No.: 17/745,715

(22) Filed: May 16, 2022

Related U.S. Application Data
(60) Provisional application No. 63/189,013, filed on May 14, 2021.

(51) Int. Cl. G16H 50/20; G06N 3/08; G16H 10/60

(52) U.S. Cl. CPC G16H 50/20; G06N 3/08; G16H 10/60

(57) ABSTRACT

Methods, systems, and apparatus, including computer programs encoded on a computer storage medium, for performing a machine learning task on a network input to generate a network output. In one aspect, one of the systems includes a neural network configured to perform the machine learning task, the neural network including one or more blocks that each include a feedforward spatial transformation unit.

### CROSS-REFERENCE TO RELATED APPLICATION

This application claims priority to U.S. Provisional Application No. 63/189,013, filed on May 14, 2021. The disclosure of the prior application is considered part of and is incorporated by reference in the disclosure of this application.

### BACKGROUND

This specification relates to performing a machine learning task on a network input using neural networks.

Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

### SUMMARY

This specification describes a system implemented as computer programs on one or more computers in one or more locations that performs a machine learning task on a network input using a neural network that includes multiple neural network blocks, at least one of which has a feedforward spatial transformation unit. A feedforward spatial transformation unit is a unit that receives as input a sequence of vectors and applies a feedforward spatial transformation that integrates information across the positions in the sequence, so that the respective spatially transformed input vector at a given position depends on the respective input vector at multiple different positions and not just the transformed input vector at the given position. A “feedforward” spatial transformation is one that does not use any recurrent or attention-based operations.

Particular embodiments of the subject matter described in this specification can be implemented so as to realize one or more of the following advantages.

This specification describes a system that performs sequential tasks, i.e., tasks that require processing a sequence of inputs, using a feedforward neural network with simple spatial interaction mechanisms and that achieves results that are comparable to or that exceed that of Transformers, which are a state-of-the-art class of sequence processing neural networks that require a complex and computationally intensive self-attention mechanism to be repeatedly applied over the input sequence.

More specifically, by removing or substantially reducing the capacity allocated to self-attention within the neural network, the described neural networks can achieve this excellent performance while consuming many fewer computational resources than Transformers and being easier to deploy on a variety of computing devices, e.g., on edge devices or within data centers, than Transformers. For example, because the operations performed by the described spatial interaction mechanism have a static parameterization (as opposed to Transformers, which use a dynamically parallelized spatial interaction mechanism), the operations can be much more readily mapped to machine learning hardware accelerators, e.g., GPUs, TPUs, or other ASICs, that are configured to perform matrix and vector multiplication in hardware. As a result, the described neural network can be readily deployed on devices that are equipped with one or more such accelerators and used to perform inference with low latency.

Moreover, the systems disclosed herein utilizing a feedforward neural network with simple spatial interaction mechanisms achieve greater performance (e.g., accuracy) for any given number of parameters/FLOPs (floating-point operations per second) than existing feedforward neural network architectures.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example neural network system.

FIG. 2 shows an example architecture of the neural network.

FIG. 3 is a flow diagram of an example process for processing an input sequence using one of the blocks in the neural network.

Like reference numbers and designations in the various drawings indicate like elements.

### DETAILED DESCRIPTION

This specification describes a system implemented as computer programs on one or more computers in one or more locations that performs a machine learning task on a network input to generate a network output for the machine learning task.

The machine learning task can be any machine learning task that operates on a network input that is an input sequence to generate a network output for the network input.

Some examples of machine learning tasks that the system can be configured to perform follow.

As another example, the task may be an audio processing task. For example, if the input to the neural network is a sequence representing a spoken utterance, the output can be a classification output that classifies the spoken utterance into one or more categories from a set of categories. For example, if the input to the neural network is a sequence representing a spoken utterance, the output generated by the neural network can indicate whether a particular word or phrase (“hotword”) was spoken in the utterance. As another example, if the input to the neural network is a sequence representing a spoken utterance, the output generated by the neural network can identify the natural language in which the utterance was spoken.

As another example, the task can be a natural language processing or understanding task, e.g., an entailment task, a paraphrase task, a textual similarity task, a sentiment task, a sentence completion task, a grammaticality task, and so on, that operates on a sequence of text in some natural language to generate classification output that classifies the text into one or more categories from a set of categories.

As another example, the task can be a health prediction task, where the input is a sequence derived from electronic health record data for a patient and the output is a prediction that is relevant to the future health of the patient, e.g., a predicted treatment that should be prescribed to the patient, the likelihood that an adverse health event will occur to the patient, or a predicted diagnosis for the patient.

As another example, the task can be an agent control task, where the input is a sequence of observations or other data characterizing states of an environment and the output defines an action to be performed by the agent in response to the most recent data in the sequence. The agent can be, e.g., a real-world or simulated robot, a control system for an industrial facility, or a control system that controls a different kind of agent.

As another example, the task can be a genomics task, where the input is a sequence representing a fragment of a DNA sequence or other molecule sequence and the output is either an embedding of the fragment for use in a downstream task, e.g., by making use of an unsupervised learning technique on a data set of DNA sequence fragments, or an output for the downstream task. Examples of downstream tasks include promoter site prediction, methylation analysis, predicting functional effects of non-coding variants, and so on.

As another example, the task can be a computer vision task, where the input is an image or a point cloud and the output is a computer vision output for the image or point cloud, e.g., a classification output that includes a respective score for each of a plurality of categories, with each score representing the likelihood that the image or point cloud includes an object belonging to the category.

When the input is an image or point cloud, the neural network can include an embedding subnetwork that generates a respective embedding for each multiple patches of the image or point cloud, and the input to the first block of the neural network can be a sequence that includes the respective embeddings (and, optionally, one or more additional embeddings, e.g., at a predetermined position that will later be used to generate the output). Each patch includes the intensity values of the pixels in a different region of the input image.

In some cases, the machine learning task is a combination of multiple individual machine learning tasks, i.e., the system is configured to perform multiple different individual machine learning tasks, e.g., two or more of the machine learning tasks mentioned above. For example, the system can be configured to perform multiple individual natural language understanding tasks, with the network input including an identifier for the individual natural language understanding task to be performed on the network input.

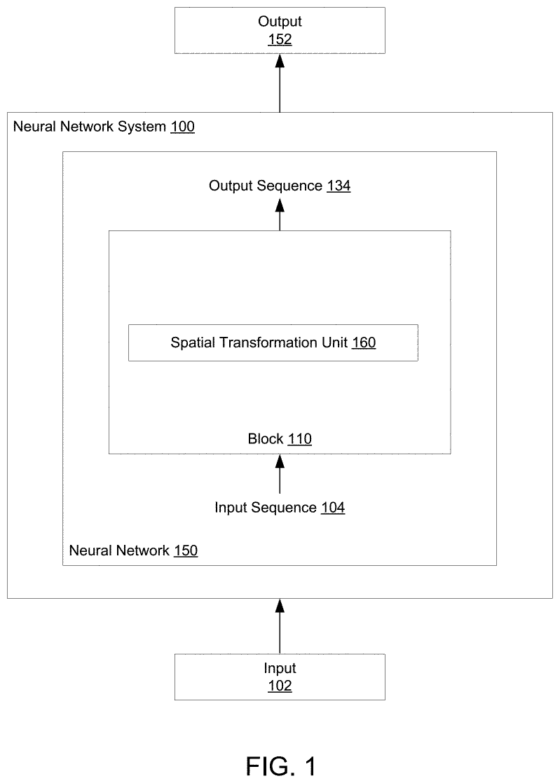

FIG. 1 shows an example neural network system 100. The neural network system 100 is an example of a system implemented as computer programs on one or more computers in one or more locations, in which the systems, components, and techniques described below can be implemented.

The neural network system 100 can receive an input 102 and perform a machine learning task on the input 102 to generate an output 152.

As described above, the neural network system 100 can perform any of a variety of tasks that involve operating on an input 102 that is an input sequence.

The neural network system 100 includes a neural network 150 that includes a sequence of multiple blocks 110. Each block implements a collection of learned operations and operates on a respective input sequence that includes a respective input vector at each of one or more positions.

That is, each block 110 operates on an input sequence 104 and generates a corresponding output sequence 134.

Specifically, the input sequence 104 has a respective input at each of a plurality of input positions in an input order and the output sequence 134 has a respective output at each of the positions in the input order. That is, the block generates a respective output for each input position in the input sequence 104.

In general, the input sequence 104 to a given block 110 can be any intermediate sequential data generated by the neural network when performing the machine learning task on the input 102.

For example, the neural network 150 can include an embedding subnetwork that includes one or more layers that generate, from the network input 102, a sequence of embeddings.

For example, when the input 102 is an image or point cloud, the neural network 150 can include an embedding subnetwork that generates a respective embedding for each of multiple patches of the image or point cloud, and the input to the first block of the neural network 150 can be a sequence that includes the respective embeddings. Each patch includes the intensity values of the pixels in a different region of the input image. For example, the embedding subnetwork can, for each patch, process the intensity values of the pixels in the patch using a learned transformation to generate the embedding of the patch.

As another example, when the input 102 is a natural language sequence, the neural network 150 can include an embedding subnetwork that generates a respective embedding for each of the text tokens in the natural language sequence, and the input to the first block of the neural network 150 can be a sequence that includes the respective embeddings. The text tokens can include, e.g., characters or other text symbols, word pieces, or words from the natural language sequence. For example, the embedding subnetwork can map each token in a vocabulary of tokens to a learned embedding for token.

Optionally, in any of the above examples, when generating the sequence of embeddings, the embedding subnetwork can append an embedding of a placeholder input, e.g., a predefined or learned “class” embedding of a predetermined “class” input that will later be used to generate the network output.

Thus, the input sequence 104 to the first block 110 in the neural network 150 includes embedded (i.e., numeric) representations of the network input 102 generated by the embedding subnetwork.

The input sequence 104 to every block 110 after the first block can be the output sequence 134 generated by the preceding block.

The neural network 150 can also include an output subnetwork that processes one or more of the vectors in the output sequence 134 generated by the last block 110 in the sequence to generate the output 152 for the machine learning task.

In implementations where the input sequence 104 to the first block 110 includes the embedding of the class input, the output subnetwork can process the vector from the output sequence 134 that corresponds to the class input, i.e., at the same position as the embedding of the class input, to generate the output 152 for the machine learning task.

In some other implementations, when the input sequence 104 to the first block 110 does not include an embedding for the class input, the output subnetwork receives the output sequence 134 generated by the last block 110 and applies a pooling operation, e.g., global average pooling, over the output vectors to generate a pooled output vector and then processes the pooled output vector to generate the output 152 for the machine learning task.

The output subnetwork can have any appropriate architecture that allows the subnetwork to map a vector to the output 152 for the machine learning task. For example, when the task is a classification task, the output subnetwork can include one or more fully-connected layers, e.g., linear layers, optionally followed by a softmax layer. When the output is a regression task, the output subnetwork can include one or more fully-connected layer followed by a different type of output layer appropriate for the regression task, e.g., a linear layer, a sigmoid output layer, and so on.

To generate the output sequence 134 from the input sequence 104, each block 110 includes a spatial transformation unit 160 that applies a feedforward spatial transformation to an input sequence to the unit that integrates information across the plurality of positions in the input sequence to the unit.

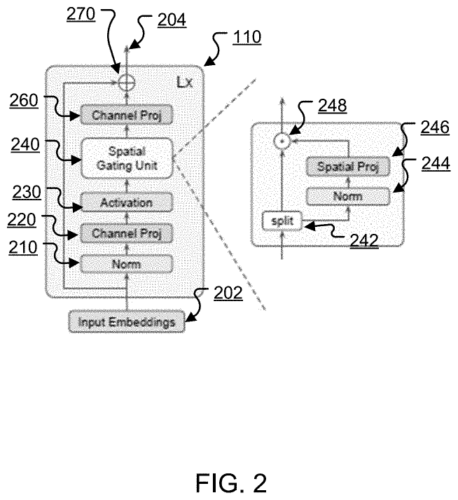

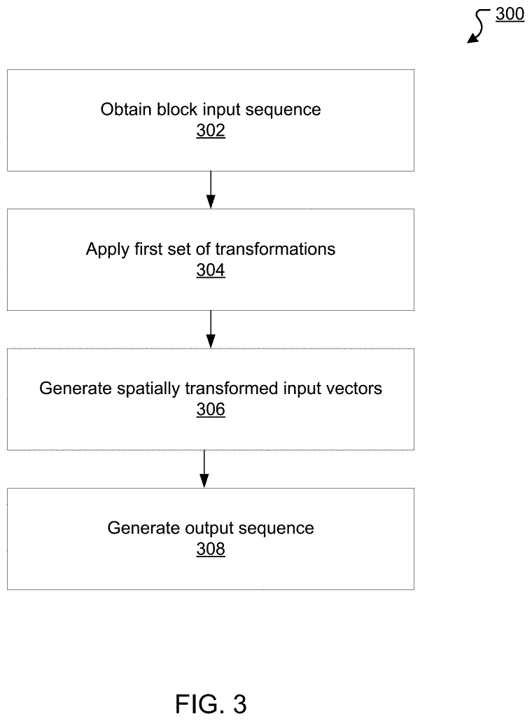

The operations performed by the blocks 110 will be described in more detail below with reference to FIGS. 2 and 3.

FIG. 2 shows an example architecture 200 for the neural network 150.

As shown in FIG. 2, the neural network 150 includes a sequence of L blocks 110.

Each block 110 obtains an input sequence for the block that includes a respective input vector at each of a plurality of positions. Each input vector has a first number d_model of channels, i.e., having d_model entries. That is, a vector that has d_model dimensions can be considered to have d_model channels, with each entry of the vector being in one of the d_model channels.

More specifically, the first block 110 in the sequence of L blocks receives as input a sequence of input embeddings 202, i.e., as generated by an embedding subnetwork as described above, that includes a respective input embedding at each of the positions.

Each block after the first block receives, as input, an output sequence that is generated by the preceding block 110.

The output sequence 204 generated by the last block 110 can, e.g., be provided as input to an output subnetwork as described above.

To generate an output sequence from an input sequence, each block 110 applies, for each position, a first set of transformations to the respective input vector at the position to generate a respective transformed input vector at the position. Each respective transformed input vector has a second number d_ffn of channels, i.e., having d_ffn entries. Generally, the second number d_ffn of channels is greater than the first number d_model of channels. The first set of transformations are applied channel-wise, i.e., so that the respective transformed input vector at a given position depends only on the respective input vector at the given position and not on any input vectors at any other positions.

As shown in the example of FIG. 2, the first set of transformations includes a normalization (“norm”) operation 210, a channel projection (“channel proj”) operation 220, and a non-linear activation (“activation”) operation 230.

That is, the block 110 applies the normalization operation 210 to the respective input vectors to generate a respective normalized input vector for each position, i.e., normalizes each input vector to generate a corresponding normalized input vector.

For each position, the block 110 then applies the channel projection operation 220 to the respective normalized input vector at the position to generate a respective initial transformed input vector for the position that has the second number of channels. In other words, the block 110 projects each normalized input vector into a higher dimensionality by applying a first projection matrix to the normalized input vector.

For each position, the block 110 applies the non-linear activation operation 230, i.e., applies an activation function, to the respective initial transformed input vector for the position to generate the respective transformed input vector for the position. The activation function can be any appropriate non-linear elementwise activation function, e.g., ReLU or GeLU.

In other words, given a matrix X that includes the vectors in the input sequence, the block 110 generates a matrix Z of the transformed input vectors as:

Z=σ((norm(X))U),

where norm(X) is the normalization operation 210, U is the first projection matrix, and σ represents the activation function.

The block 110 then generates a respective spatially transformed input vector at each of the positions using a spatial transformation unit that applies a feedforward spatial transformation that integrates information across the plurality of positions. That is, the spatial transformation unit applies a feedforward spatial transformation that integrates information across the plurality of positions, so that the respective spatially transformed input vector at a given position depends on the respective transformed input vector at multiple different positions and not just the transformed input vector at the given position. A feedforward spatial transformation is one that does not use any recurrent or attention-based operations.

In other words, given the matrix Z of the transformed input vectors, the block 110 generates a matrix \tilde{Z} of the spatially transformed input vectors as \tilde{Z}=s(Z), where s represents the operations of the spatial transformation unit.

In the example of FIG. 2, the spatial transformation unit is a spatial gating unit 240 that, in addition to applying the feedforward spatial transformation, also applies a gating mechanism as part of generating the spatially transformed input vectors.

More specifically, FIG. 2 shows one example of the operations performed by the spatial gating unit 240.

In the example of FIG. 2, the spatial gating unit 240 applies a split operation 242 to generate, for each position, a respective first partial vector that includes a first subset of the second number of channels of the respective transformed input vector for the position and a respective second partial vector that includes a second subset of the second number of channels of the respective transformed input vector for the position. That is, the unit 240 “splits” each transformed input vector into two along the channel dimension. For example, one partial vector can include the first half of the channels and the other partial vector can include the other half of the channels.

The spatial gating unit 240 then applies a normalization 244 to the respective first partial vectors to generate a respective normalized first partial vector for each position, i.e., normalizes each first partial vector.

The spatial gating unit 240 then applies, to the respective normalized first partial vectors, a feedforward spatial transformation 246 (“spatial proj”) that combines information across the respective normalized first partial vectors at the positions to generate a respective spatially transformed partial vector for each position.

As a particular example, the unit 240 can determine a product between (i) a spatial transformation matrix and (ii) a matrix of the respective normalized first partial vectors and then add a bias term to the product to generate the respective spatially transformed partial vectors for each position.

The spatial gating unit 240 then generates the respective spatially transformed input vector at each of the positions from at least the respective spatially transformed partial vectors and the respective second partial vectors. For example, the spatial gating unit 240 can perform a gating between the respective spatially transformed partial vectors and the respective second partial vector for the positions. That is, for each position, the unit 240 can determine an element-wise product 248 between (i) the respective spatially transformed partial vector for the position and (ii) the respective second partial vector for the position.

In other words, in this example, s(Z)=Z₁⊙ƒ_{W,b}(norm(Z₂)), where ƒ_{W,b}(norm(Z₂))=W(norm(Z₂))+b, Z₁ is a matrix of the second partial vectors, Z₂ is a matrix of the first partial vectors, ⊙ represents element-wise multiplication, norm(Z₂) represents a normalization operation, W is the spatial transformation matrix, and b is the bias term.

In some implementations, at the outset of training, a training system can initialize Was near-zero values, e.g., to randomly selected values that are within a threshold distance of zero, and b as ones, meaning that ƒ_{W,b}(norm(Z₂)) is approximately equal to one and s(Z) is approximately equal to Z₁ at the beginning of training. Initializing these values in this way can ensures that each block behaves like a regular FFN (feedforward neural network) that does not mix information across vectors at the early stage of training and only gradually injects spatial information across tokens during the course of learning. This can improve the stability of the training process.

Thus, in some implementations (including the implementation shown in FIG. 2), the unit 240 (and, more generally, the neural network) does not include any self-attention operations.

State-of-the-art Transformers neural networks typically perform the spatial aggregation of information across inputs in a sequence using a multi-head self-attention mechanism. “Self-attention” refers to an operation that aggregates spatial information across tokens in a sequence by, for each token, applying attention over all of the tokens in the sequence to generate the updated token at the position. Multi-head self-attention performs multiple instances of this operation in parallel and then combines the outputs of each to generate the final output of the operation. This type of spatial aggregation is dynamically parameterized based on the input representations, i.e., the weights in the weighted sum for each position are dependent on how many inputs there are in the input sequence and on the values in each of the input vectors. Self-attention and, in particular, multi-head attention can consume a significant amount of the computational capacity of a Transformer neural network.

By replacing the multi-head self-attention with the spatial transformation described above, the neural network 150 can achieve results that are comparable to or that exceeding that of Transformer neural networks while consuming many fewer computational resources and being easier to deploy on a variety of computing devices, e.g., on edge devices or within data centers.

In some other implementations, the unit 240 incorporates a self-attention mechanism when generating the respective spatially transformed input vector at each of the positions. In particular, in these implementations, the unit 240 can apply a self-attention mechanism to the input sequence for the block to generate a respective attended input vector at each of the positions and then, for each position, determine a sum between (i) the respective spatially transformed partial vector for the position and (ii) the respective attended input vector for the position to generate a respective combined vector for the position. The unit 240 can then determine an element-wise product between (i) the respective combined vector for the position and (ii) the respective second partial vector for the position to generate the spatially transformed vectors.

Thus, in order to apply a self-attention mechanism to the input sequence for the block, the unit 240 can optionally first normalize the input sequence and then apply a linear transformation to generate a respective query, key, and value for each input vector in the input sequence. The unit 240 then generates, for each position, a respective attention weight for each position in the sequence from the query for the position and the keys for all of the positions and then computes a weighted sum of the values for the positions, i.e., with each value weighted by the corresponding attention weight. Optionally, if the dimensionality of the queries, keys, and values is too large, the unit 240 can then project each weighted sum to have a dimensionality equal to the spatially transformed partial vectors.

Including the self-attention mechanism can improve the performance of the neural network on certain tasks, e.g., on natural language tasks that require processing long sequences of text that includes multiple different sentences and aligning information between text that is located in different sentences.

Even when the block 110 includes a self-attention mechanism, because of the presence of the unit 240, the self-attention mechanism can be a “tiny” mechanism that is significantly more computationally efficient than the attention mechanisms that are employed by state-of-the-art Transformer neural networks. For example, the self-attention mechanism can be a single-head mechanism instead of the multi-head mechanism employed by Transformer neural networks. Additionally, the dimensionality of the queries, keys, and values can be significantly smaller than the dimensionality employed by Transformer neural networks. In other words, even when the block includes a self-attention mechanism, each block 110 nonetheless consumes significantly fewer computational resources than a given block in a state-of-the art Transformer neural network.

Generally, the example of FIG. 2 describes implementations where the unit 240 “splits” the transformed input vectors. In some other implementations, however, the unit 240 does not perform this “splitting” and instead operates directly on the transformed input vectors.

In these implementations, the unit 240 can apply a normalization to the respective transformed input vectors to generate a respective normalized transformed input vector for each position and then apply, to the respective transformed input vectors, a feedforward spatial transformation that combines information across the respective transformed input vectors at the positions to generate a respective spatially transformed vector for each position.

For example, applying, to the respective transformed input vectors, a feedforward spatial transformation can include determining a product between (i) a spatial transformation matrix and (ii) a matrix of the respective transformed input vectors and adding a bias term to the product to generate the respective spatially transformed vectors for each position.

The unit 240 can then generate the respective spatially transformed input vector at each of the positions from at least the respective spatially transformed vectors and the respective transformed input vectors. For example, the unit 240 can, for each position: determine an element-wise product between (i) the respective spatially transformed vector for the position and (ii) the respective transformed input vector for the position.

When the unit 240 also applies self-attention, the unit 2140 can apply a self-attention mechanism to the input sequence for the block to generate a respective attended input vector at each of the positions; and for each position: determining a sum between (i) the respective spatially transformed vector for the position and (ii) the respective attended input vector for the position to generate a respective combined vector for the position; and determine an element-wise product between (i) the respective combined vector for the position and (ii) the respective transformed input vector for the position.

The block 110 then generates the output sequence for the block 110 by, for each position, applying a second set of transformations to the respective spatially transformed input vector at the position to generate a respective output vector at the position. The second set of transformations are applied channel-wise, i.e., so that the output vector at a given position depends only on the respective spatially transformed input vector at the given position and not on any respective spatially transformed input vectors at any other positions.

In the example of FIG. 2, the second set of transformations includes a channel projection (“channel proj”) operation 260 and a skip connection 270.

That is, applying a second set of transformations to the respective input vector at the position includes applying the channel projection operation 260, i.e., applying a second projection matrix to the respective spatially transformed input vector at the position to generate a respective initial output vector for the position having the first number of channels, i.e., to decrease the number of channels from d_ffn or, when the “split” operation is included, from half of (or a different fraction of) d_ffn, to d_model.

Applying the second set of transformations then includes applying the skip connection 270 to the respective initial output vector at each position by adding the respective initial output vector for the position to the respective input vector to the position to generate the respective output vector for the position.

Thus, as described above with reference to FIG. 2, each block 110 first projects each input to a higher dimensionality, i.e., a higher number of channels, and then applies the feedforward spatial transformation in the higher dimensional space, and then projects the output of the feedforward spatial transformation back to the original dimensionality.

In other words, a matrix Y of the output vectors in the output sequence satisfies:

Y=X+\tilde{Z}V,

where V is the second projection matrix.

As can be seen from the above, when self-attention is not included, the parameterization of the spatial transformation is static, i.e., is not dependent on the input and is fixed after the neural network is trained. That is, the projection matrices have a fixed dimensionality and are fixed after training completes. To allow for fixed size matrices, when the system operates on variable length data, e.g., when the input sequences are variable length, the system can pad the data with zero vectors or garbage vectors so that each sequence operated on by the neural network 150 has a fixed size. For example, the system can pad the original network input to the system with zero vectors or the embedding subnetwork can append a pre-determined embedding after the embeddings for the inputs in the network input to generate a fixed length sequence of embeddings.

Because the spatial projection matrices have a fixed dimensionality, the spatial transformations preserve positional information throughout the neural network. Therefore, unlike Transformer neural networks, the embeddings generates by the embedding subnetwork do not need to encode positional information, i.e., information that identifies the position of a given embedding within the sequence of the embedding. In other words, the embeddings in the sequence of embeddings are not generated using any positional embeddings or any other positional information, further simplifying the architecture of the neural network 150.

FIG. 3 is a flow diagram of an example process 300 for processing an input sequence using one of the blocks in the neural network. For convenience, the process 300 will be described as being performed by a system of one or more computers located in one or more locations. For example, a neural network system that includes a sequence of blocks, e.g., neural network system 100 of FIG. 1, appropriately programmed in accordance with this specification, can perform the process 300.

During the processing of a network input by the neural network, the block obtains an input sequence for the block that includes a respective input vector at each of a plurality of positions, with each input vector having a first number d_model of channels, i.e., having d_model, entries (step 302).

For each position, the block applies a first set of transformations to the respective input vector at the position to generate a respective transformed input vector at the position, each respective transformed input vector having a second number d_ffn of channels, i.e., having d_ffn entries (step 304). Generally, the second number of channels is greater than the first number of channels. The first set of transformations are applied channel-wise, i.e., so that the respective transformed input vector at a given position depends only on the respective input vector at the given position and not on any input vectors at any other positions.

The block then generates a respective spatially transformed input vector at each of the positions (step 306). Generating the spatially transformed input vectors includes applying a feedforward spatial transformation that integrates information across the plurality of positions, so that the respective spatially transformed input vector at a given position depends on the respective transformed input vector at multiple different positions and not just the transformed input vector at the given position. A feedforward spatial transformation is one that does not use any recurrent or attention-based operations.

The block then generates an output sequence for the block by, for each position, applying a second set of transformations to the respective spatially transformed input vector at the position to generate a respective output vector at the position (step 308). The second set of transformations are applied channel-wise, i.e., so that the output vector at a given position depends only on the respective spatially transformed input vector at the given position and not on any respective spatially transformed input vectors at any other positions.

Prior to using the neural network to perform the machine learning task, a training system trains the neural network to perform the task, i.e., to determine trained values of the parameters of the neural network, i.e., of the blocks in the sequence, the output subnetwork, and the embedding subnetwork. For example, the training system can train the neural network from scratch on training data for the task to minimize a loss function for the task, e.g., a cross-entropy loss, a negative log likelihood loss, and so on using conventional machine learning techniques. As another example, the training system can first pre-train the mixer neural network on an unsupervised objective and then fine-tune the mixer neural network on the training data for the task. As yet another example, the training system can train the mixer neural network on both unlabeled data and the training data for the task through semi-supervised learning.

During training, the training system can incorporate any number of techniques to improve the speed, the effectiveness, or both of the training process. For example, the system can use dropout, label smoothing, or both to reduce overfitting. As another example, the system can perform the training using a distributed architecture that trains multiple instances of the neural network in parallel. Moreover, as described above, the system can first pre-train the neural network on a large unsupervised data set through unsupervised learning, e.g., to minimize a BERT loss or other unsupervised loss, and then fine-tune the neural network on task-specific training data to optimize the loss function for the task.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software- based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute- intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A system for performing a machine learning task on a network input to generate a network output, the system comprising one or more computers and one or more storage devices storing instructions that, when executed by the one or more computers, cause the one or more computers to implement:
a neural network configured to perform the machine learning task, the neural network comprising a plurality of blocks, each block configured to perform operations comprising:
obtaining an input sequence for the block comprising a respective input vector at each of a plurality of positions, each input vector having a first number of channels;
for each position, applying a first set of transformations to the respective input vector at the position to generate a respective transformed input vector at the position, each respective transformed input vector having a second number of channels;
generating a respective spatially transformed input vector at each of the positions, comprising applying a feedforward spatial transformation that integrates information across the plurality of positions; and
generating an output sequence for the block by, for each position, applying a second set of transformations to the respective spatially transformed input vector at the position to generate a respective output vector at the position.

2. The system of claim 1, wherein the neural network further comprises:
one or more output layers configured to process one or more of the respective output vectors in the output sequence for a last block of the plurality of blocks to generate the network output.

3. The system of claim 1, wherein applying the spatial transformation comprises:
for each position, generating a respective first partial vector that includes a first subset of the second number of channels of the respective transformed input vector for the position and a respective second partial vector that includes a second subset of the second number of channels of the respective transformed input vector for the position;
applying a normalization to the respective first partial vectors to generate a respective normalized first partial vector for each position;
applying, to the respective normalized first partial vectors, a feedforward spatial transformation that combines information across the respective normalized first partial vectors at the positions to generate a respective spatially transformed partial vector for each position; and
generating the respective spatially transformed input vector at each of the positions from at least the respective spatially transformed partial vectors and the respective second partial vectors.

4. The system of claim 3, wherein applying, to the respective normalized first partial vectors, a feedforward spatial transformation comprises:
determining a product between (i) a spatial transformation matrix and (ii) a matrix of the respective normalized first partial vectors; and
adding a bias term to the product to generate the respective spatially transformed partial vectors for each position.

5. The system of claim 3, wherein generating the respective spatially transformed input vector at each of the positions comprises, for each position:
determining an element-wise product between (i) the respective spatially transformed partial vector for the position and (ii) the respective second partial vector for the position.

6. The system of claim 3, wherein generating the respective spatially transformed input vector at each of the positions comprises:
applying a self-attention mechanism to the input sequence for the block to generate a respective attended input vector at each of the positions; and
for each position:
determining a sum between (i) the respective spatially transformed partial vector for the position and (ii) the respective attended input vector for the position to generate a respective combined vector for the position; and
determining an element-wise product between (i) the respective combined vector for the position and (ii) the respective second partial vector for the position.

7. The system of claim 1, wherein applying the spatial transformation comprises:
applying a normalization to the respective transformed input vectors to generate a respective normalized transformed input vector for each position;
applying, to the respective transformed input vectors, a feedforward spatial transformation that combines information across the respective transformed input vectors at the positions to generate a respective spatially transformed vector for each position; and
generating the respective spatially transformed input vector at each of the positions from at least the respective spatially transformed vectors and the respective transformed input vectors.

8. The system of claim 7, wherein applying, to the respective transformed input vectors, a feedforward spatial transformation comprises:
determining a product between (i) a spatial transformation matrix and (ii) a matrix of the respective transformed input vectors; and
adding a bias term to the product to generate the respective spatially transformed vectors for each position.

9. The system of claim 7, wherein generating the respective spatially transformed input vector at each of the positions comprises, for each position:
determining an element-wise product between (i) the respective spatially transformed vector for the position and (ii) the respective transformed input vector for the position.

10. The system of claim 7, wherein generating the respective spatially transformed input vector at each of the positions comprises:
applying a self-attention mechanism to the input sequence for the block to generate a respective attended input vector at each of the positions; and
for each position:
determining a sum between (i) the respective spatially transformed vector for the position and (ii) the respective attended input vector for the position to generate a respective combined vector for the position; and
determining an element-wise product between (i) the respective combined vector for the position and (ii) the respective transformed input vector for the position.

11. The system of claim 1, wherein for each position, applying a first set of transformations to the respective input vector at the position comprises:
applying a normalization to the respective input vectors to generate a respective normalized input vector for each position;
for each position, applying a first projection matrix to the respective normalized input at the position to generate a respective initial transformed input vector for the position having the second number of channels; and
for each position, applying an activation function to the respective initial transformed input vector for the position to generate the respective transformed input vector for the position.

12. The system of claim 1, wherein for each position, applying a second set of transformations to the respective input vector at the position comprises:
applying a second projection matrix to the respective spatially transformed input vector at the position to generate a respective initial output vector for the position having the first number of channels.

13. The system of claim 12, wherein for each position, applying a second set of transformations to the respective input vector at the position comprises:
adding the respective initial output vector for the position to the respective input vector to the position to generate the respective output vector for the position.

14. The system of claim 1, wherein the input sequence for a first block of the plurality of blocks is a sequence of embeddings that represent the network input.

15. The system of claim 14, wherein the embeddings are not generated using any positional embeddings.

16. The system of claim 14, wherein the network input is an image, and wherein the sequence of embeddings comprises a respective embedding representing each of a plurality of patches from the image.

17. The system of claim 1, wherein the machine learning task operates on a network input that is an input sequence to generate a network output for the network input, and the machine learning task comprises:
an audio processing task, wherein the network input is a sequence representing a spoken utterance, and the network output is a classification output that classifies the spoken utterance into one or more categories from a set of categories;
a health prediction task, wherein the network input is a sequence derived from electronic health record data for a patient, and the network output is a predicted diagnosis for the patient;
an agent control task, wherein the network input is a sequence of observations or other data characterizing states of an environment, and the network output defines an action to be performed by the agent in response to the most recent data in the sequence;
a genomics task, wherein the network input is a sequence representing a fragment of a DNA sequence or other molecule sequence, and the network output is either an embedding of the fragment for use in a downstream task or an output for the downstream task; or
a computer vision task, wherein the network input is an image or a point cloud and the output is a computer vision output for the image or point cloud.

18. The system of claim 1, wherein the machine learning task is an image classification task, the network input is an image, and the network output is a classification output that includes a respective score for each of a plurality of categories, with each score representing the likelihood that the image includes an object belonging to the category.

19. One or more computer-readable storage media storing instructions that when executed by one or more computers cause the one or more computers to implement:
a neural network configured to perform the machine learning task, the neural network comprising a plurality of blocks, each block configured to perform operations comprising:
obtaining an input sequence for the block comprising a respective input vector at each of a plurality of positions, each input vector having a first number of channels;
for each position, applying a first set of transformations to the respective input vector at the position to generate a respective transformed input vector at the position, each respective transformed input vector having a second number of channels;
generating a respective spatially transformed input vector at each of the positions, comprising applying a feedforward spatial transformation that integrates information across the plurality of positions; and
generating an output sequence for the block by, for each position, applying a second set of transformations to the respective spatially transformed input vector at the position to generate a respective output vector at the position.

20. A method performed by one or more computers, the method comprising:
receiving a network input; and
processing the network input using a neural network to generate a network output for the network input, wherein the neural network comprises a plurality of blocks, and wherein the processing comprises, for each block:
obtaining an input sequence for the block comprising a respective input vector at each of a plurality of positions, each input vector having a first number of channels;
for each position, applying a first set of transformations to the respective input vector at the position to generate a respective transformed input vector at the position, each respective transformed input vector having a second number of channels;
generating a respective spatially transformed input vector at each of the positions, comprising applying a feedforward spatial transformation that integrates information across the plurality of positions; and
generating an output sequence for the block by, for each position, applying a second set of transformations to the respective spatially transformed input vector at the position to generate a respective output vector at the position.
